(19) United States
(12) Patent Application Publication
(10) Pub. No.: US 2002/0182593 A1
(43) Pub. Date: Dec. 5, 2002

(54) Strawberry vein banding virus (SVBV) promoter

(75) Inventors: Gusui Wu, Davis, CA (US); Kevin McBride, Davis, CA (US)

Correspondence Address: MAXYGEN, INC., 515 GALVESTON DRIVE, RED WOOD CITY, CA, 94063, US

(73) Assignee: Maxygen, Inc., Redwood City, CA

(21) Appl. No.: 10/020,540

(22) Filed: Oct. 30, 2001

Related U.S. Application Data
(60) Provisional application No. 60/245,354, filed on Nov. 1, 2000.

(51) Int. Cl. C12Q 1/70; C07H 21/04; A01H 1/00; C12N 5/04; A01H 5/00

(52) U.S. Cl. 435/5; 800/280; 435/320.1; 435/419; 536/23.6

(57) ABSTRACT

The present invention provides promoters for driving the expression of heterologous polynucleotide sequences in plant cells. The promoters are derived from strawberry vein banding virus (SVBV), and include variants, derivatives, fragments, and modified versions of the naturally occurring promoter sequences. The present invention also provides expression cassettes comprising the promoters, as well as methods of using the promoters to drive gene expression in plant cells and transgenic plants.

### CROSS REFERENCE TO RELATED APPLICATIONS

This application claims the benefit under 35 U.S.C. §119(e) of U.S. Provisional Application No. 60/245,354, filed Nov. 1, 2000, the disclosure of which is incorporated by reference for all purposes.

### COPYRIGHT NOTIFICATION PURSUANT TO 37 C.F.R. §1.71(E)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention is in the field of plant genetic engineering. Specifically, the present invention provides promoter sequences that are particularly useful in driving the expression of heterologous polynucleotide sequences in plants.

### BACKGROUND OF THE INVENTION

Promoters that give strong and constitutive gene expression are critical tools for plant genetic engineering. For example, they are used to introduce numerous desirable traits into plants, as well as for studying gene function. The most widely used constitutive promoters in plants are those isolated from caulimoviruses, also called plant pararetroviruses.

Caulimoviruses have a small genome consisting of double-stranded circular DNA. The viral genome is replicated via reverse transcription of the terminally redundant full-length RNA transcripts by a viral reverse transcriptase. Two major RNA transcripts are also made from the viral DNA by the host DNA-dependent RNA polymerase II. In cauliflower mosaic virus (CaMV), the type member of Caulimoviridae, the two transcripts are the 35S and the 19S RNAs. The promoter of the 35S RNA transcripts resides in the large intergenic region between ORFs VI and VII. Promoter sequences of this region isolated from CaMV and figwort mosaic virus (FMV) have been shown to function in many plants, and are widely used for heterologous gene expression in plants. Enhancer elements have been identified within the region and have been shown to have synergistic effects on the activity of the viral promoters by duplication.

SVBV is a member of the caulimovirus family and has been sub-grouped together with CaMV and FMV. The genome of SVBV Strain E3 has recently been sequenced (Petrzik et al., (1998) Virus Genes, 16:303-305). In the SVBV genome, there are seven putative genes, represented by ORFs I to VII, and two regulatory intergenic regions, which are responsible for the transcription of the full-length RNA transcript and the small RNA transcript. The sequence similarities in the regulatory regions between SVBV and CaMV or FMV are rather low (30-45%).

There is a need for new plant promoters useful in driving transgenes in transgenic plants. The present invention addresses these and other needs.

### SUMMARY OF THE INVENTION

The present invention provides promoters for driving the expression of heterologous polynucleotide sequences in plant cells. The promoters are derived from strawberry vein banding virus (SVBV), e.g., SVBV Strain E3, and include variants, derivatives, fragments, and modified versions of the naturally occurring promoter sequences. The present invention also provides expression cassettes comprising the promoters, as well as methods of using the promoters to drive gene expression in plant cells and transgenic plants.

As such, in one aspect, the present invention provides an isolated or recombinant nucleic acid molecule comprising a promoter operably linked to a heterologous polynucleotide, wherein the promoter comprises a nucleotide sequence that is at least about 70% identical to a nucleotide sequence selected from the group consisting of SEQ ID NOS:1-4. In some embodiments of the invention, the promoter comprises a nucleotide sequence that is at least about 75%, 80%, 85%, 90%, 95%, 96%, 97%, 98%, 99%, 99.5% or 99.9% identical to a nucleotide sequence selected from the group consisting of SEQ ID NOS:1-4.

The invention further provides an isolated or recombinant nucleic acid molecule comprising a promoter operably linked to a heterologous polynucleotide, wherein the promoter comprises a nucleotide sequence that is at least 90% identical to 100, or preferably at least 90% identical to 200, 300, 400 or 500 contiguous nucleotides in a nucleotide sequence selected from SEQ ID NOS: 1-4.

The invention further provides an isolated or recombinant nucleic acid molecule comprising a promoter operably linked to a heterologous polynucleotide, wherein the promoter comprises at least 20, or preferably at least 40, 60, 80, 100, 200, 300, 400 or 500 contiguous nucleotides in a nucleotide sequence selected from SEQ ID NOS: 1-4.

In one embodiment, a strand of the promoter specifically hybridizes to a nucleic acid molecule comprising a nucleotide sequence selected from the group consisting of SEQ ID NOS:1-4, or a complement thereof. In another embodiment, the promoter comprises a nucleotide sequence selected from the group consisting of SEQ ID NOS:1-4. In another embodiment, the promoter is derived from SVBV, e.g., SVBV Strain E3. In another embodiment, the nucleic acid further comprises a heterologous polynucleotide, operably linked to said promoter. In another embodiment, the heterologous polynucleotide encodes a polypeptide. In another embodiment, expression of said heterologous polynucleotide produces an antisense or sense RNA for gene suppression. In another embodiment, the nucleic acid molecule is a plasmid suitable for transfection of a plant cell. In another embodiment, the plasmid comprises a selectable marker gene with or without Agrobacteriuni border sequences. In another embodiment, the promoter comprises two or more enhancer elements. In another embodiment, the promoter is chimeric. A chimeric promoter of the invention can comprise, for example a minimal promoter region derived from SVBV, an enhancer element derived from SVBV, or two or more enhancer element derived from SVBV.

In another aspect, the present invention provides an expression cassette comprising an SVBV promoter comprising a nucleotide sequence that is at least about 70% identical to a nucleotide sequence selected from SEQ ID NOS: 1-4, operably linked to a heterologous polynucleotide.

In one embodiment, the promoter specifically hybridizes to a nucleic acid molecule comprising the nucleotide sequence of a nucleotide sequence selected from SEQ ID NOS: 1-4 or a complement thereof. In another embodiment, the promoter comprises the nucleotide sequence of a nucleotide sequence selected from SEQ ID NOS: 1-4. In another embodiment, the promoter is derived from SVBV Strain E3. In another embodiment, the heterologous polynucleotide encodes a polypeptide. In another embodiment, expression of said heterologous polynucleotide produces an antisense or sense RNA for gene suppression. In another embodiment, the expression cassette further comprises an enhancer element. In another embodiment, the expression cassette further comprises a transcription termination signal sequence.

In another aspect, the present invention provides a host cell transfected with any of the above-described expression cassettes.

In one embodiment, the cell is a plant cell. In another embodiment, the cell is present within a plant.

In another aspect, the present invention provides a transgenic plant comprising any of the above-described expression cassettes.

In one embodiment, the plant is a monocot. In another embodiment, the plant is a dicot. Some exemplary plants provided by the invention include Arabidopsis, tobacco, cotton, corn and soybean.

In another aspect, the present invention provides a method of expressing a heterologous polynucleotide in a plant cell, the method comprising: (i) providing an expression cassette comprising the heterologous polynucleotide, operably linked to an SVBV promoter of the invention; and (ii) introducing the expression cassette into a host cell, whereby the heterologous polynucleotide is expressed.

In one embodiment, the promoter specifically hybridizes to a nucleic acid molecule comprising the nucleotide sequence of a nucleotide sequence selected from SEQ ID NOS: 1-4. or a complement thereof. In another embodiment, the promoter comprises the nucleotide sequence of a nucleotide sequence selected from SEQ ID NOS: 1-4. In another embodiment, the heterologous polynucleotide encodes a polypeptide. In another embodiment, expression of the heterologous polynucleotide in the plant cell produces an antisense RNA. In another embodiment, the plant cell is present within a plant. In another embodiment, the expression cassette is introduced into the cell using Agrobacterium.

### DESCRIPTION OF THE INVENTION AND PREFERRED EMBODIMENTS

I. Introduction

This invention provides full-length RNA transcript promoter sequences of SVBV. In addition, the invention provides truncated, modified, shuffled and chimeric derivatives of these promoters, and the use of the promoters for gene expression in plants and plant cells. The promoter sequences can be modified to improve their properties in any of a number of ways, such as to increase the level or tissue specificity of expression of polynucleotide sequences operably linked to the promoter. Such modifications include the insertion of multiple copies of the enhancer element of the SVBV promoter, insertion of one or more copies of an enhancer element (or multiple enhancer elements) derived from one or more different promoters, as well as the shuffling of the promoter sequence or part of the promoter sequence. The use of any of the herein-described promoters can be used to drive foreign gene expression in plant cells, including protoplasts, cell suspensions, tissues, and transgenic plants. Any polynucleotide sequence can be expressed, including sequences encoding any protein of interest, antisense sequences, sense sequences, ribozymes, and others.

The herein-provided SVBV promoters typically provide strong and constitutive expression in plant cells of genes under their control. As such, they can serve as an alternative to other constitutive promoters such as the 35S promoter of CaMV and the 34S promoter of FMV. Further, by modifying or shuffling the regulatory elements of the promoters, it is possible to generate promoters with novel tissue specificity and improved strength that are useful for the genetic engineering of a wide variety of traits in plants.

II. Definitions

The terms “polynucleotide,” “nucleotide sequence,” and “nucleic acid” are used to refer to a polymer of niucleotides (A,C,T,U, etc. or naturally occurring or artificial nucleotide analogues), e.g., DNA or RNA, or a representation thereof, e.g., a character string, etc, depending on the relevant context. A given polynucleotide or complementary polynucleotide can be determined from any specified nucleotide sequence.

The term “promoter” refers to a region of DNA upstream from the translational start codon and which is-involved in recognition and binding of RNA polymerase and other proteins to initiate transcription. A “plant promoter” is a promoter capable of initiating transcription in plant cells. The terms “SVBV plant promoter” or “SVBV promoter” as used herein refer to plant promoters derived from the promoter region of an SVBV gene. A promoter of the invention might include only some portion of an SVBV-derived promoter (e.g., a chimeric promoter), so long as that portion is intrinsically has promoter activity, or the portion in the context of surrounding heterologous sequence has promoter activity.

The terms “constitutive promoter or constitutive plant promoter” as used herein refer to a plant promoter which is capable of expressing operably linked DNA sequences continuously, typically in all tissues or nearly all tissues of a plant during normal development. The terms “inducible promoter” or inducible plant promoter, as used herein, refer to plant promoters that are capable of selectively expressing operably linked DNA sequences at particular times in response to endogenous or external stimuli.

The term “tissue-specific promoter” as used herein refers to promoters that are capable of selectively expressing operably linked DNA sequences in particular tissues, e.g., particular plant tissues. This means that the expression of the operatively linked DNA sequences is higher in one or several plant tissues than it is in the other tissues of the plant. This tissue specificity can also depend on the developmental stage or environmental conditions of the plant, for example driving expression in a particular set of tissues at one developmental stage, and in another set of tissues at a later stage.

The term “operably linked” as used herein refers to linkage of a promoter upstream from a DNA sequence such that the promoter mediates transcription of the DNA sequence. It is understood that the promoter sequence also includes transcribed sequences between the transcriptional start and the translational start codon.

The phrase “expression cassette” refers to nucleotide sequences that are capable of affecting expression of a structural gene in hosts compatible with such sequences. Such cassettes include at least promoters and optionally, transcription termination signals. Additional factors necessary or helpful in effecting expression may also be used as described herein.

A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. In the case of Agrobacterium vectors, the polynucleotide will include a T-DNA expression cassette for introduction of a desired nucleic acid into plant cells.

The term “plasmid” refers to an autonomous circular DNA molecule capable of replication in a cell, and includes both the expression and nonexpression types. Where a recombinant microorganism or cell culture is described as hosting an “expression plasmid”, this includes both extrachromosomal circular DNA molecules and DNA that has been incorporated into the host chromosome(s). Where a plasmid is being maintained by a host cell, the plasmid is either being stably replicated by the cells during mitosis as an autonomous structure or is incorporated within the host's genome.

The phrase “heterologous sequence” or a “heterologous DNA sequence”, as used herein, is one that originates from a foreign source (or species) or, if from the same source, is modified from its original form or is not normally found in association with a sequence of interest. Thus, a heterologous DNA-encoding sequence operably linked to a promoter is from a source different from that from which the promoter was derived, or, if from the same source, is modified from its original forn or is normally operably linked to a different promoter. Modification of the heterologous DNA sequence may occur, e.g., by treating the DNA with a restriction enzyme to generate a DNA fragment that is capable of being operably linked to the promoter. Modification can occur by techniques such as site-directed mutagenesis and others well known to those of skill in the art. Preferred means of modifying the promoters of the invention are described below.

The phrase “nucleic acid sequence encoding” refers to a nucleic acid that directs the expression of a specific protein or peptide. The nucleic acid sequences include both the DNA strand sequence that is transcribed into RNA and the RNA sequence that is translated into protein. The nucleic acid sequences include both the full-length nucleic acid sequences as well as non-full-length sequences derived from the full-length protein. It being further understood that the sequence includes the degenerate codons of the native sequence or sequences that mav he introduced to provide codon preference in a specific host cell.

The phrase “isolated” or “substantially pure” when referring to SVBV promoter sequences refer to isolated nucleic acids that contain promoter sequences from an SVBV gene, but which do not contain promoter sequences from other genes. A polynucleotide is “isolated” when it is partially or completely separated from components with which it-is normally associated (other proteins, nucleic acids, cells, synthetic reagents, etc.). A nucleic acid or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “plant” includes whole plants, plant organs (e.g., leaves, stems, roots, etc.), seeds, plant cells as well as protoplasts and cell suspensions. The class of plants which can be used in the method of the invention is generally as broad as the class of higher plants amenable to transformation techniques, including both monocotyledonous and dicotyledonous plants. It includes plants of a variety of ploidy levels, including polyploid, diploid and haploid.

The term “transgenic plant” refers to a plant that has been produced by genetic engineering techniques. For example, plant cells transformed with vectors containing SVBV promoters operably linked to heterologous DNA sequences can be used to produce transgenic plants with altered phenotypic characteristics.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For example, a test nucleotide sequence is 70% identical to a reference nucleotide sequence if, when the two sequences are aligned for maximum percent identity, at least 70% of the nucleotides in the reference sequence are paired (i.e., aligned) with the identical nucleotide in the test sequence. If the test sequence is shorter than the reference sequence, then any nucleotides at the 5′ and/or 3′ end of the reference sequence that are not aligned with a corresponding nucleotide in the test sequence are counted as mismatches. So, for example, where the first 90 nucleotides of a 100 nucleotide reference sequence is aligned with a 90 nucleotide test sequence, the percent identity if all 90 nucleotides match is 90%.

The phrase “substantially identical,” in the context of two nucleic acids or polypeptides, refers to two or more sequences or subsequences that have at least about 60%, usually about 70%, about 75%, about 80%, about 85%, about 90%, or about 95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, (1981) Adv. Appl. Math. 2:482, by the homology alignment algorithm of Needleman & Wunsch, (1970) J. Mol. Biol. 48:443, by the search for similarity method of Pearson & Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally, Current Protocols in Molecular Biology, F. M. Ausubel et al., eds., Current Protocols, a joint venture between Greene Publishing Associates, Inc. and John Wiley & Sons, Inc., (1995 Supplement) (“Ausubel”)).

Examples of algorithms that are suitable for determining percent sequence identity and sequence similarity BLAST and BLAST 2.0 algorithms, which are described in Altschul et al. (1990) J. Mol. Biol. 215:403-410 and Altschuel et al. (1977) Nucleic Acids Res. 25:3389-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff, (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences are substantially identical is that the two molecules hybridize to each other under stringent conditions, as described below.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 3° C. for short probes (e.g., 10 to 50 nucleotides) and at least about 60° C. for long probes (e.g., greater than 50 nucleotides). Stringent conditions may also be achieved with the addition of destabilizing agents such as formamide.

III Preparation of plant SVBV promoters

This invention relates to isolated plant SVBV promoters and recombinant DNA constructs containing the SVBV promoter. These DNA constructs include expression cassettes and a variety of vectors.

Nucleic acid sequences corresponding to plant SVBV promoters are typically identical to or show substantial sequence identity (determined as described above) to the nucleic acid sequences depicted in SEQ ID NOS:1-4. SVBV promoter sequences will typically hybridize to the nucleic acid sequence of one or more of SEQ ID NOS:1-4, or complementary sequences thereof, under stringent conditions. For example, high stringency hybridization can be done in buffer containing 50% formamide, 10% dextran sulfate, 10×Denhardts, 100 μg/ml salmon sperm DNA, 1% SDS, 50mM NaPO₄, and 0.6M NaCl at 42° C. Filters are subsequently washed at 65° C. in 0.1×SSC, 0.1% SDS. Other stringent hybridization conditions may also bc selected. Generially, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents and the extent of base mismatching, the combination of parameters is more important than the absolute measure of any one.

Techniques for nucleic acid manipulation such as subcloning promoter sequences into expression vectors, labeling probes, DNA hybridization, and the like are described generally in Sambrook, et al., (1989) Molecular Cloning—A Laboratory Manual (2nd Ed.), Vol. 1-3, Cold Spring Harbor Laboratory, Cold Spring Harbor, New York (“Sambrook”).

SVBV promoters can be either synthesized or isolated in any of a number of ways. For example, DNA is isolated from a genomic library using labeled probes having sequences complementary to the sequences disclosed herein (e.g., SEQ ID NOS:1-4). Full-length probes may be used, or oligonucleotide probes may be designed that are complementary to any portion of SEQ ID NOS:1-4. Such probes can be used directly in hybridization assays to isolate DNA corresponding to SVBV promoters. Alternatively, probes can be designed for use in amplification techniques such as PCR. In addition, a nucleic acid can be isolated from a genomic library using an SVBV coding sequence, and the sequence upstream of the coding sequence can be identified as an SVBV promoter. Other methods known to those of skill in the art can also be used to isolate plant DNA fragments containing Gp2 promoters. See, e.g., Sambrook, et al. for a description of other techniques for the isolation of DNAs related to DNA molecules of known sequence.

To prepare a genomic library, typically the DNA is extracted from plant tissue and either mechanically sheared or enzymatically digested to yield fragments of about 15-20 kb. In one embodiment, the fragments are separated by gradient centrifugation from undesired sizes and are constructed in vectors, e.g., bacteriophage lambda vectors. These vectors and phage are packaged in vitro, as described in Sambrook. Recombinant phage are analyzed by plaque hybridization as described in Benton and Davis. (1977) Science 196:180-182. Colony hybridization is carried out as generally described in M. Grunstein, et al. (1975) Proc. Natl. Acad. Sci. USA., 72:3961-3965. DNA corresponding to SVBV promoters is identified in genomic libraries by its ability to hybridize with nucleic acid probes, for example on Southern blots, and these DNA regions are isolated by standard methods familiar to those of skill in the art. See, e.g., Sambrook.

Nucleic acid amplification techniques such as polymerase chain reaction (PCR) technology can be used to amplify nucleic acid sequences corresponding to SVBV promoters from genomic libraries. In PCR techniques, oligonucleotide primers complementary to the two borders of the DNA region to be amplified are synthesized. The polymerase chain reaction is then carried out using the two primers. See, e.g., (1990) PCR Protocols: A Guide to Methods and Applications (Innis, et al., eds.), Academic Press, San Diego. Primers can be selected to amplify the entire SVBV promoter. PCR can also be used to amplify smaller DNA segments of a full-length promoter as desired.

Oligonucleotides for use as probes in the above-mentioned procedures can be chemically synthesized according to the solid phase phosphoramidite triester method first described by Beaucage & Carruthers (1981; Tetrahedron Lett. 22:1859-1862) using an automated synthesizer, as described in Needham-VanDevanter, et al., (1984; Nucleic Acids Res., 12:6159-6168). Purification of oligonucleotides is by either native acrylamide gel electrophoresis or by anion-exchange HPLC as described in Pearson, J. D. and Regnier, F. E. (1983; J. Chrom. 255:137-149). The sequence of the synthetic oligonucleotide can be verified using the chemical degradation method of Maxam & Gilbert (1980), in Grossman, L. and Moldave, D., eds. Academic Press, New York, Methods in Enzymology, 65:499-560.

Once a nucleic acid comprising an SVBV promoter sequence has been isolated, different forms of the promoter can be produced. In many embodiments, the different forms of the promoter have different properties, as longer sequences may contain additional elements such as enhancers, repressors, etc. that may affect the level, regulation, or tissue specificity of expression. SVBV promoters are typically from about 100 to about 2000 nucleotides in length (e.g., 0.4, 0.8, or 1.4 kb). SVBV promoters of different sizes can be constructed in a variety of ways known to those of skill in the art. For example, shorter forms of the promoters can be constructed by mapping restriction enzyme sites in the promoter and then using the constructed map to determine appropriate restriction enzyme cleavage to excise a subset of the sequence. The shorter restriction fragment can then he inserted into a suitable vector. Alternatively, primers can be designed for PCR to amplify a promoter sequence of a desired length.

In one aspect the invention provides a minimal SVBV promoter. A mininmal promoter will typically comprise a TATA box and transcriptional start sequence, but will not contain additional stimulatory and repressive elements. Typically the boundaries defining the minimal promoter sequence are to some extent ambiguous. That is, depending upon the-expression reporter system used and the threshold level of observed expression required for a determination that a promoter is functional, the nucleotide positions defining the 5′and 3′ends of a minimal promoter might vary slightly. An exemplary plant minimal promoter is located at around nucleotide positions −50 to +8 of the 35S CaMV promoter. Exemplary animal minimal promoters include the SV40 early minimal promoter and the CMV promoter from positions −53 to +75 (Gossen, et al. Proc. Natl. Acad. Sci. USA 89:5547 (1992)). A fungal minimal promoter includes the Saccharomycetes cerevisiae iso-1-cytochrome c (cycl) promoter, as well as the GALI promoter. A bacterial minimal promoter includes the lacZ minimal promoter. The boundaries defining the SVBV minimal promoter can be readily determined by one of skill in the art using known techniques, described, for example, in Fang et al. (1989) Plant Cell 1:141-50 and Odell et al. (1985) Nature 313:810-2.

The invention further provides modified forms of the SVBV promoter wherein some or all of the SVBV promoter sequence is duplicated. This duplication can provides enhanced promoter activity. For example providing two or more copies of an enhancer element in tandem often results in increased expression levels. The methodology for generating such improved promoters is known in the art, and described, for example, in the following publications: Kay et al. (1987) Science 236:1299-1302; Odell et al. (1988) Plant Molecular Biology 10:263-72; Dey and Maiti (1999) Transgenic Research 3:61-70; Maiti et al. (1997) Transgenic Research 6:143-56; and Maiti and Shepard (1998) Biochem Biophys Res Commun 244:440-44. Using the methodology described in these papers, or variations of these methods readily implemented by the skilled artisan, one can obtain any number of enhanced variants of the SVBV promoters described herein.

The invention further provides chimeric promoters comprising some portion of an SVBV promoter as described herein. For example, a chimeric promoter comprising an SVBV minimal promoter combined with an enhancer element (or multiple enhancer elements) derived from a different (i.e., heterologous) promoter can be used. Another example would be a combination of an SVBV enhancer element with a minimal promoter derived from another promoter sequence. The portion of a chimeric promoter of the invention that is not derived from SVBV promoter can be derived from another plant promoter, from a viral promoter, or from any other naturally-occurring promoter. Alternatively, that portion can be entirely synthetic, or a modified variation of a naturally-occurring promoter. An example of a chimeric promoter is described in Comai et al. (1990) Plant Mol Biol 15:373-81. Similar methodology can be employed in generating a chimeric promoter of the invention.

Some exemplary enhanced SVBV promoter variants were generated using this methodology and shown to promote increased levels of expression, as described in the Examples. By providing two or more enhancer elements in tandem, it is

An SVBV minimal promoter of the invention can be used alone or in combination with

The type and level of expression of a polynucleotide sequences operably linked to any of there herein-described SVBV promoters can be readily assessed. For example, a vector can be prepared that has a particular SVBV promoter operably linked to an indicator gene. Plant cells are then transformed with the vector and transgenic plants are produced from the transformed plant cells. Expression of the indicator gene under the control of the SVBV promoter is then determined.

IV. Modification—SVBV promoters

Modifications can be made to the SVBV promoter to, for example, either strengthen its activity or generate novel tissue-specificity. A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for the ability to direct expression of a desired operably linked nucleic acid. Following diversification by one or more of the methods herein, or otherwise available to one of skill, any nucleic acids that are produced can be selected for a desired activity or property. This can include identifying any activity that can be detected, for example, in an automated or automatable format, by any of the assays in the art, e.g., by assaying the hydrolysis of carboxymethyl-chitin-remazol brilliant violet, as described herein. A variety of related (or even unrelated) properties can be evaluated, in serial or in parallel, at the discretion of the practitioner.

Descriptions of a variety of diversity generating procedures for generating modified SVBV promoter sequences of the invention are found the following publications and the references cited therein: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Clang et al. (1999) “Evolution of a cytokine using DNA family shuffling”Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechriques 18:194 195; Stemmer et al., (1995) “Single-step assembly of a gene and entire plasmid form large numbers of oligodeoxy-ribonucleotides” Gene, 164:49-53; Stemmer (1995) “The Evolution of Molecular Computation”Science 270:1510; Stemmer (1995) “Searching Sequence Space” BioTechnology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2):157-178; Dal “Oligonucleotide-directed random mutagenesis using the phosphorothioate method” Methods Mol. Biol. 57:369-374; Smith (1985) “In vitro mutagenesis” Ann. Rev. Genet. 19:423-462: Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed inutagenesis (Metiods in Enzymol. 100:468-500 (1983); Methods in Enzymol. 154:329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13:8749-8764; Taylor et al. (1985) “The rapid generation of oligonucleotide-directed mutations at high frequency using phosphorothioate-modified DNA” Nucl. Acids Res. 13:8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14:9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16:803-814); mutagenesis using gapped duplex DNA (Kramer et al. (1984) “The gapped duplex DNA approach to oligonucleotide-directed mutation construction” Nucl. Acids Res. 12:9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16:7207; and Fritz et al. (1988) “Oligonucleotide-directed construction of mutations: a gapped duplex DNA procedure without enzymatic reactions in vitro” Nucl. Acids Res. 16:6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13:4431-4443; and Carter (1987) “Improved oligonucleotide-directed mutagenesis using M13 vectors” Methods in Enzymol. 154:382-403), deletion mutagenesis (Eghtedarzadeh & Henikoff (1986) “Use of oligonucleotides to generate large deletions” Nucl. Acids Res. 14:5115), restriction-selection and restriction-selection and restriction-purification (Wells et al. (1986) “Importance of hydrogen-bond formation in stabilizing the transition state of subtilisin” Phil. Trans. R. Soc. Lond. A 317:415-423), mutagenesis by total gene synthesis (Nambiar et al. (1984) “Total synthesis and cloning of a gene coding for the ribonuclease S protein” Science 223:1299-1301; Sakamar and Khorana (1988) “Total synthesis and expression of a gene for the a-subunit of bovine rod outer segment guanine nucleotide-binding protein (transducin)” Nucl. Acids Res. 14:6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13:3305-3316), double-strand break repair (Mandecki (1986); Arnold (1993) “Protein engineering for unusual environments” Current Opinion in Biotechnology 4:450-455. “Oligonuclcotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzyniology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Craineri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41368 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/13487 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold at. al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al.,“Method for Constructing a Library Using DNA Shuffling,” and WO 98/42727 by Pati and Zarling, “Sequence Alterations using Homologous-Recombination.”

Certain U.S. applications provide additional details regarding various diversity generating methods, including “Shuffling of Codon Altered Genes” by Patten et al. filed Sep. 28, 1999, (U.S. Ser. No. 09/407,800); “Evolution of Whole Cells and Organisms by Recursive Sequence Recombination”, by del Cardayre et al. filed Jul. 15, 1998 (U.S. 09/166,188), and Jul. 15, 1999 (U.S. 09/354,922); “Oligonucleotide Mediated Nucleic Acid Recombination” by Crameri et al., filed Sep. 28, 1999 (U.S. Ser. No. 09/408,392), and “Oligonucleotide Mediated Nucleic Acid Recombination” by Craineri et al., filed Jan. 18, 2000 (PCT/US00/01203); “Use of Codon-Based Oligonucleotide Synthesis for Synthetic Shuffling” by Welch et al., filed Sep. 28, 1999(U.S. Ser. No. 09/408,393); “Methods for Making Character Strings, Polynucleotides & Polypeptides Having Desired Characteristics” by Selifonov et al., filed Jan. 18, 2000,(PCT/US00/01202) and, e.g., “Methods for Making Character Strings, Polynucleotides & Polypeptides Having Desired Characteristics” by Selifonov et al., filed Jul. 18, 2000,(U.S. Ser. No. 09/618,579); “Methods of Populating Data Structures for Use in Evolutionary Simulations” by Selifonov and Stemmer (U.S. Ser. No. PCT/US00/01138), filed Jan. 18, 2000; and “Single-Stranded Nucleic Acid Template-Mediated Recombination and Nucleic Acid Fragment Isolation” by Affholter, U.S. Ser. No. 60/186,482, filed Mar. 2, 2000.

In brief, several different general classes of sequence modification methods, such as mutation, recombination, etc. are applicable to the present invention and set forth, e.g., in the references above. That is the SVBV promoter nucleic acids of the invention can be generated from wild type sequences. Moreover, the promoter sequences of the invention can be modified to create modified sequences with the same or different activity.

The following exemplify some of the different types of preferred formats for diversity generation in the context of the present invention, including, e.g., certain recombination based diversity generation fonnats.

Nucleic acids can be recombined in vitro by any of a variety of techniques discussed in the references above, including e.g., DNAsc digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751. Thus, for example, nucleic acids encoding chitinase with modified activity can be generated.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “Oligonucleotide Mediated Nucleic Acid Recombination” by Cramer et al., filed Sep. 28, 1999 (U.S. Ser. No. 09i408,392), and “Oligonucleotide Mediated Nucleic Acid Recombination” by Crameri et al., filed Jan. 18, 2000 (PCT/US00/01203); “Use of Codon-Based Oligonucleotide Synthesis for Synthetic Shuffling” by Welch et al., filed Sep. 28, 1999 (U.S. Ser. No. 09/408,393); “Methods for Making Character Strings, Polynucleotides & Polypeptides Having Desired Characteristics” by Selifonov et al., filed Jan. 18, 2000, (PCT/US00/01202); “Methods of Populating Data Structures for Use in Evolutionary Simulations” by Selifonov and Stemmer (PCT/US00/01138), filed Jan. 18, 2000; and, e.g., “Methods for Making Character Strings, Polynucleotides & Polypeptides Having Desired Characteristics” by Selifonov et al., filed Jul. 18, 2000 (U.S. Ser. No. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “Methods for Making Character Strings, Polynucleotides & Polypeptides Having Desired Characteristics” by Selifonov et al., filed Jan. 18, 2000, (PCT/US00/01202) “Methods of Populating Data Structures for Use in Evolutionary Simulations” by Selifonov and Stemmer (PCT/US00/0138), filed Jan. 18, 2000; and, e.g., “Methods for Making Character Strings, Polynucleotides & Polypeptides Having Desired Characteristics” by Selifonov et al., filed Jul. 18, 2000 (U.S. Ser. No. 09/618,579). Extensive details regarding in silico recombination methods are found in these applications. This methodology is generally applicable to the present invention in providing for recombination of the chitinase nucleic acids in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full-length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, U.S. Ser. No. 60/186,482, filed Mar. 2, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalcnt chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeler et al. (1999) “Combinatorial Protein Engineering by Incremental Truncation,” Proc. Natl. Acad. Sci. USA, 96:3562-67; Ostermeier et al. (1999), “Incremental Truncation as a Strategy in the Engineering of Novel Biocatalysts,” Biological and Medicinal Chenistry, 7:2139-44.

Mutational methods which result in the alteration of individual nucleotides or groups of contiguous or non-contiguous nucleotides can be favorably employed to introduce nucleotide diversity. Thus, modified chitinase nucleic acids of the invention can be generated, including for optimized codon usage for an organism of interest, as well as nucleic acids encoding chitinase polypeptides with improved and/or modified activity. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genoine, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods for Generating and Screening Novel Metabolic Pathways,” and Thompson, et al. (1998) U.S. Pat. No. 5,824,485 Methods for Generating and Screening Novel Metabolic Pathways) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA librarya normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215:471) prior to diversifying according to any of the methods described herein.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well.

It will readily be appreciated that any of the above described techniques suitable for enriching a library prior to diversification can also be used to screen the products, or libraries of products, produced by the diversity generating methods.

Kits for mutagenesis, library construction and other diversity gencration methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International pic (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

The modified promoters made by the methods described above can be tested for desired activities using standard techniques. A general method for selecting desired promoters comprises introducing the reassembled promoter into a basal or minimal promoter construct that is operably linked to a reporter gene. By testing constructs of the invention for reporter gene activity under desired conditions and cell types, a reassembled polynucleotide that confers an improved or desired transcriptional activity can be determined. Selection of cell or organisms to test the constructs of the invention is determined by the desired promoter activity.

In some embodiments, particularly where a high-expression promoter is desired, an organism (e.g., a plant) is transformed with candidate reassembled promoters operably linked to GFP and transformants are analyzed for fluorescence in tissues where promoter activity os desired. In other embodiments where specific tissue expression is desired in a seed of a plant, plant lines with clear seed coats are selected (e.g., tt mutants in Arabidopsis) and candidate promoters operably linked to a visual marker (e.g., GFP, lycopene, β-carotene, etc.) are transformed into such plants. Seed harvested from the primary transformants with seed-specific promoters are recognized by a change of color in the seed.

Similarly, fruit-specific promoters can be identified in tomato fruit by operably linking the GFP gene to promoter candidates and transforming tomato. A particularly useful variety of tomato for this procedure is a “microtom” variety.

Reporter genes are generally useful for analyzing the transcriptional activity of a candidate promoter. Reporter genes are operably linked to a candidate promoter and then expressed. The protein encoded by the reporter gene typically produces a detectable product which can be compared visually or analytically (e.g., by ELISA). Alternatively, the quantity of the product can be determined by measuring light absorbance at a specific wavelength of a sample comprising the sample. Examples of reporter systems include luciferase (Cohn et al., Proc. Natl. Acad. Sci. USA 80:102-123 (1983); U.S. Pat. No. 5,196,524), β-galactosidase (Jefferson, et al., Proc. Natl. Acad. Sci. USA 83:8447-8451 (1986)), β-glucuronidase (GUS) (GUS PROTOCOLS: USING THE GUS GENE AS A REPORTER OF GENE EXPRESSION (ed. Gallagher) Academic Press, New York 1992) and green fluorescent protein (see, e.g., U.S. Pat. Nos. 5,491,084 and 5,958,713). These and other reporter genes are useful in screening large numbers (e.g., a library) of promoter variants or putative promoters. These sorts of screening procedures can be used, for example, in conjunction with promoter sequence diversification (achieved using, e.g., mutagenesis, recombination, gene shuffling, and the like, as described herein) to identify promoter variants with altered or improved finctional characteristics.

V. Construction of Vectors Containing an SVBV Promoter Operably Linked to a Heterologous DNA Sequence

Once a plant SVBV promoter region has been isolated, and a particular size, variant, or derivative (e.g., shuffled derivative) of the promoter has been selected, various methods may be used to construct various forms of the promoter. These different forms can then be used in expression cassettes, vectors and other DNA constructs. A variety of techniques can be used for these manipulations of nucleic acids. These techniques are known to those of skill in the art and are described generally in Sambrook, et al., spra.

Expression cassettes containing an SVBV promoter can be constructed in a variety of ways. For instance, an SVBV promoter sequence can be amplified using a primer sequence that includes a restriction site at the 3′ end of the promoter sequence, e.g., at the normal SVBV transcription start site. In this way, heterologous sequences can be introduced into the vector such that the expression of the heterologous sequences is regulated by the promoter. DNA constructs composed of an SVBV promoter operably linked to heterologous DNA sequences can then be inserted into a variety of vectors, such as expression vectors that are useful in the transformation of plant cells. Such vectors can be constructed by the use of recombinant DNA techniques well known to those of skill in the art. The vectors will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to glyphosate, chlorosulfuron or Basta. The expression vector may also at least one enhancer element and/or a transcription termination signal sequence.

An expression cassette or vector of the invention preferably includes a 3′ non-translated DNA sequence that encodes a polyadenylation signal that functions in a host cell (e.g., a plant cell) to cause the termination of transcription and the addition of polyadenylate nucleotides to the 3′ end of the transcribed RNA. The expression cassette or vector will also preferably include downstream of the promoter a 5′ non-translated leader sequence. In one embodiment, the 5′ end of the non-translated sequence is the naturally occurring end of SVBV promoter, and the 3′ end of the non-translated sequence is optimized for translation initiation (Kozak (1986) Nature 315:200-04).

VI. Production of Transgenic Plants

A. Heterologous DNA sequences

DNA constructs containing an SVBV promoter operably linked to a heterologous DNA sequence can be used to transform plant cells and produce transgenic plants. In one embodiment, a protein-encoding polynucleotide sequence is operably linked to an SVBV promoter and used to transform plant cells. Transgenic plants can be produced from the transformed plant cells so that the gene product is produced in all tissues or in only certain tissues of a transgenic plant. Such methods are useful for any of a number of applications, including but not limited to, compensating for missing or altered gene expression in a plant, expressing an endogenous protein at higher than normal levels, or expressing a novel gene in a plant. The expression of the novel gene can result in the production of a protein that confers an altered phenotype on a transgenic plant.

DNA constructs containing an SVBV promoter operably linked to a heterologous DNA sequence can also be used in a number of techniques to suppress expression of endogenous plant genes, e.g., sense or antisense suppression. In antisense technology, a nucleic acid segment from the desired plant gene is cloned and operably linked to an SVBV promoter such that the anti-sense strand of RNA will be transcribed. The construct is then transformed into plants and the anti-sense strand of RNA is produced. In plant cells, it has been shown that anti-sense RNA inhibits gene expression; see, e.g., Sheehy, et al., (1988) Proc. Nat. Acad. Sci. USA, 85:8805-8809, and Hiatt, et al., U.S. Pat. No. 4,801,340.

The nucleic acid segment to be introduced in antisense suppression generally will be substantially identical to at least a portion of the endogenous gene or genes to be repressed, but need not be identical. The vectors of the present invention can be designed such that the inhibitory effect applies to other members of a family of genes exhibiting homology or substantial homology to the target gene.

The introduced sequence also need not be full-length relative to either the primary transcription product or fully processed MRNA. Generally, higher homology can be used to compensate for the use of a shorter sequence. Furthermore, the introduced sequence need not have the same intron or exon pattern, and homology of non-coding segments will be equally effective. Normally, a sequence of between about 30 or 40 nucleotides and about 2,000 nucleotides should be used, though a sequence of at least about 100 nucleotides is preferred, a sequence of at least about 200 nucleotides is more preferred, and a sequence of at least about 500 nucleotides is especially preferred.

Catalytic RNA molecules or ribozymes also have been reported to have use as a means to inhibit expression of endogenous plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyine sequences within antisense RNAs confers RNA-cleaving activity upon them, thereby increasing the activity of the constructs.

A number of classes of ribozymes have been identified. One class of ribozyme is derived from a number of small circular RNAs which are capable of self-cleavage and replication in plants. Thc RNAs replicate cithcr alonc (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described, for example, in Haseloff, et al. (1988) Nature, 334:585-591.

A preferred method of suppression is sense suppression. Introduction of a nucleic acid configured in the sense orientation has been shown to be an effective means by which to block the transcription of target genes; see, e.g., Napoli, et al., (1990) The Plant Cell 2:279-289, and U.S. Pat. No. 5,034,323.

Generally, in sense suppression, some transcription of the introduced sequence occurs. The effect may also occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity is useful to exert a more effective repression of expression of the endogenous sequences. Substantially greater identity of more than about 80% is preferred, though about 95% to absolute identity would be most preferred. The effect may be applied to other proteins within a similar family of genes exhibiting homology or substantial homology. Segments from a gene can be used (1) directly to inhibit expression of homologous genes in different plant species, or (2) as a means to obtain the corresponding sequences, which can be used to suppress the gene.

In sense suppression, the introduced sequence, needing less than absolute identity, also need not be full-length, relative to either the primary transcription product or fully processed mRNA. A higher identity in a shorter than full-length sequence compensates for a longer, less identical sequence. Furthermore, the introduced sequence need not have the same intron or exon pattern, and identity of non-coding segments may be equally effective. A sequence of a size of at least 50 base pairs is preferred, with greater length sequences being more preferred; see U.S. Pat. No. 5,034,323.

B. Transforming plant cells and plants

Techniques for transforming a wide variety of higher plant species are well known and described in the technical and scientific literature. See, for example, Weising et al. (1988) Ann. Rev. Genet. 22:421-477. DNA constructs containing an SVBV promoter linked to heterologous DNA can be introduced into the genome of the desired plant host by a variety of conventional techniques. For example, the DNA construct may be introduced directly into the genomic DNA of the plant cell using techniques such as electroporation and microinjection of plant cell protoplasts. Alternatively, the DNA constructs can be introduced directly to plant tissue using ballistic methods, such as DNA particle bombardment. In addition, the DNA constructs may be combined with suitable T-DNA flanking regions and introduced into a conventional Agrobacteriuni tumefaciens host vector. The virulence functions of the Agrobacterium tumefaciens host will direct the insertion of the construct and adjacent marker into the plant cell DNA when the cell is infected by the bacteria.

Microinjection techniques are known in the art and are well described in the scientific and patent literature. The introduction of DNA constructs using polyethylene glycol precipitation is described in Paszkowski, et al., (1984) EMBO J. 3:2717-2722. Electroporation techniques are described in Fromm et al., (1985) Proc. Natl. Acad. Sci. USA 82:5824. Ballistic transformation techniques are described in Klein et al., (1987) Nature 327:70-73.

Agrobacterium tumefaciens-meditated transformation techniques are well described in the scientific literature. See, for example Horsch, et al. (1984) Science 233:496-498, and Fraley, et al. (1983) Proc. Nail. Acad. Sci. USA 80:4803. More specifically, a plant cell, an explant, a meristem or a seed is infected with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch, etal., (1984) Science, 233:496-498; Fraley et al., (1983) Proc. Nat'l. Acad. Sci. U.S.A. 80:4803.

Ti plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causiig genes so that they no longer interfere, the modified Ti plasmid can then be used as a vector for the transfer of the gene constructs of the invention into an appropriate plant cell, such being a “disabled Ti vector”.

All plant cells which can be transformed by Agrobacterium and whole plants regenerated from the transformed cells can also be transformed according to the invention so as to produce transformed whole plants which contain the transferred foreign nucleic acid sequence. Numerous ways have been developed for the transformation of plant cells with Agrobacterium, including:

(1) co-cultivation of Agrobacterium with cultured isolated protoplasts,

(2) transformation of cells or tissues with Agrobacterium, or

(3) transformation of seeds, apices or meristems with Agrobacterium.

Method (1) requires an established culture system that allows culturing protoplasts and plant regeneration from cultured protoplasts. Method (2) requires (a) that the plant cells or tissues can be transformed by Agrobacterium and (b) that the transformed cells or tissues can be induced to regenerate into whole plants. Method (3) requires micropropagation.

In the binary system, to have infection, two plasmids are needed: a T-DNA containing plasmid and a vir plasmid. Any one of a number of T-DNA containing plasmids can be used, the only requirement is that one be able to select independently for each of the two plasmids.

After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance or visual observation. Other phenotypic markers are known in the art and may be used in this invention.

The present invention embraces use of the claimed promoters in transformation of any plant, including both dicots and monocots. Transformation of dicots is described in references above. Transformation of monocots is known using various techniques including electroporation (e.g., Shimamoto et al., (1992) Nature, 338:274-276; ballistics (e.g., European Patent Application 270,356); and Agrobacterium (e.g., Bytebier, et al., Proc. Nat'l Acad. Sci. USA (1987) 84:5345-5349).

Transformed plant cells which are derived by any of the above transformation techniques can bc cultured to regenerate a whole plant. Such regeneration techniques rely on manipulation of certain phytohonnones in a tissue culture growth medium, typically relying on a biocide and/or herbicide marker which has been introduced together with the Ph nucleotide sequences. Plant regeneration from cultured protoplasts is described in Evans et al., (1983) Protoplasts Isolation and Culture, Handbook of Plant Cell Culture, pp. 124-176, MacMillan Publishing Company, New York; and Binding, (1985) Regeneration of Plants, Plant Protoplasts, pp. 21-73, CRC Press, Boca Raton. Regeneration can also be obtained from plant callus, explants, organs, or parts thereof. Such regeneration techniques are described generally in Klee et al. (1987) Ann. Rev. Plant Phys. 38:467-486.

One of skill will recognize that, after an expression cassette comprising an SVBV promoter is stably incorporated in transgenic plants and confirmed to be operable, it can be introduced into other plants by sexual crossing. Any of a number of standard breeding techniques can be used, depending upon the species to be crossed.

The expression of the heterologous DNA sequences linked to an SVBV promoter can be detected in a variety of ways, depending on the nature of heterologous sequences. For example, one may assay for a desired phenotype. The desired phenotype which results from the successful expression of heterologous DNA sequences under control of an SVBV promoter may be determined by a variety of ways, depending on the phenotypic trait that is introduced. For instance, resistance to a herbicide can be detected by treatment with the herbicide.

Expression of the heterologous DNA can also be detected by measurement of the specific RNA transcription product. This can be done by, for example, RNAse protection or Northern blot procedures. If heterologous DNA sequences encode a novel protein, the protein product may be assayed, for instance, by its function or by a variety of immunoassay techniques. For example, a novel protein product with enzymatic activity can be measured in an enzyme assay.

The methods and compositions of the invention have use over a broad range of types of plants, including species from the genera Fragaria, Lotus, Medicago, Onobrych is, Trifolium, Trigonella, Vigna, Citrus, Linum, Geranium, Manihot, Daucus, Arabidopis, Brassica, Raphanus, Sinapis, Atropa, Capsicum, Hyoscyamus, Lycopersicon, Nicotiana, Solanum, Petunia, Digitalis, Majorana, Ciohorium, Helianthus, Lactuca, Bromus, Asparagus, Antirrhinum, Herecocallis, Neniesia, Pelargoniuni, Panicum, Pennisetum, Ranunculus, Senecio, Salpiglossis, Cucumis, Browaalia, Glycine, Loliun, Zea, Triticum, Sorghum, Datura, Chrysantheum, Dianthus, Gerbera, Euphorbia, Iponoea, Passiflora, Cyclamen, Malus, Prunus, Rosa, Rubus, Populus, Santalum, Allium, Lilium, Narcissus, Ananas, Arachis, Phaseolus and Pisum, and more particularly including oil crops such as canola (Brassica sp.), cotton (Gossypium sp.), peanut (Arachis sp.), sunflower (Helianthus sp.), palm (Elaeis sp.), flax (Linum sp.), safflower (Carthamus sp.), coconut (Cocos ap.) and soybean (Glycine sp.); grain crops such as wheat (Triticum sp.), corn (Zea sp.), sorghum (Sorghum sp.), barley (Hordeum sp.), rye (Secale sp.), oats (Avena sp.) and rice (Oryza sp.); fruit crops such as banana (Musa sp.), citrus (Citrus sp.), berries (e.g., strawberry (Fragaria Sp.) or raspberry (Rubus sp.), mango (Mangifera sp.), melon (Cucumis sp.), pear (Pyrus sp.), cucumber (Cucumis sp.), and apricot, peach, cherry, plum and prune (Prunus sp.); vegetable crops such as pea (Pisum sp.), bean (Vicia sp.), broccoli and related crucifers (Brassica sp.), spinach (spinacia sp.), onion (Allium sp.), celery (Apium sp.), carrot (Daucus sp.), asparagus (Asparagus sp.), and artichoke (Helianthus sp.); tomato (Lycopersicon esculentum), pepper (Capiscum annuum); additional ornamental crops such as tulip (Tulipa sp.), snapdragon (Antirrhinum sp.), Iris (Iris sp.), Orchids (Cymbidium and Cattleya sp.), pelargonium; beverage crops such as coffee (Coffea sp.) and tea (Thea sp.); herb crops such as mint (Mentha sp.), thyme (Thymus sp.) and marjoram (Origanum sp.).

The following examples are provided by way of illustration and not limitation.

VII. Examples

A. Isolation of the SVBV promoter

An SVBV promoter fragment was isolated by PCR amplification using oligonucleotide primers (5′CAAAGAAGAAGCTTAACTATGC3′ and 5′TGCGGGATCCGGTTTGTAAGCAGGGAGG3′) specific to sequences flanking the promoter region. Using this method, a 0.4 Kb promoter fragment consisting of sequences 6816 to 7291 of the SVBV-E3 genome was isolated (SEQ ID NO:1). The 3′ end of the promoter fragment contains the un-translated leader sequences of ORF VII to ensure translatability of coding regions located downstream.

B. Generation of reporter fusion constructs

All of the constructs created for the analysis of the various promoters contain the following elements in the stated order: a T-DNA right border region (GenBank #M63056 bp 81-199) from A. tumefaciens Ti plasmid pTiAB3, a test promoter, a test reporter gene, an Arabidopsis UBQ3 3′ polyadenylation signal (GenBank #L05363 bp 2652-3059) and a T-DNA left border region (GenBank #AE007925 bp 9407-9557) from A. tumefaciens C58. A Luciferase reporter was used, derived from plasmid pGLP3-Basic (Promega) with the exception that an AscI site had been added to the 3′ end by subcloning this gene as an HindIII to XbaI fragment into pNEB193 (New England Biolabs). A modified version of the cycle3 GFP gene (Crameri et al. (1996) Nat. Biotechnol.14:315-19), was also used as a reporter in some experiments. The cycle3 GFP gene encodes a GFP molecule that folds better at higher temperatures and thus accumulates to higher levels at temperatures above 30 degrees centigrade

Three variations of the SVBV promoter were tested: the SVBV promoter (SEQ ID NO:2), the seSVBV promoter (SEQ ID NO:3), and the leSVBV promoter (SEQ ID NO: 4). The seSVBV promoter differs from SVBV as follows: bp 1-108 of SVBV was replaced by bp 92-362 of SVBV. Likewise, promoter leSVBV differs from SVBV in that bp 1-108 of SVBV was replaced by bp 92-404 of SVBV.

The dMMV promoter from Kentucky was derived from plasmid pKM24 (Dey and Maiti (1999) Transgenic Research 3:61-70) by blunt end cloning the HindIII/EcoRt promoter fragment into the Smal site of pBCSK+(Stratagene; La Jolla, Calf.) such that the 5′ end bears a BamiHI site and the 3′ end bears a PstI site. The 35S promoter was derived from pB1121 (Clonetech; Palo Alto. Calif.) by PCR amplification with primers 5′CACACGGATCCAGAAGACCAAAGGGCAATTGAGAC3′ (SEQ ID NO:5) and 5′ACAAACTGCAGAGAGAGACTGGTGATTCAGCGTGTCCTCTCCAAATGAAA TGAACTTCCTTAT3′ (SEQ ID NO:6). These primers amplify an approximately 400 bp fragment such that a BamHI site is placed at the 5′ end and a PstI site at the 3′ end of the promoter. The d35S promoter was derived from pCAMBIA1305.1 (CAMBIA; Canberra, Australia). Primers for PCR amplification of the d35S promoter are 5′ACACAGGATCCGCGTATTGGCTAGAGCAGCTTGCC 3′ (SEQ ID NO: 7) and 5′TCTCTCCATGGAGAGATAGATTGTAGAGAGAGACTGGTG 3′ (SEQ ID NO:8). Amplification with these primers results in the addition of a BamHI site at the 5′ end of the promoter and an NcoI site at the 3′ end of the promoter.

Originally a T-DNA expression vector was created having a right border followed by a BamHI site, the SVBV promoter, PstI, NcoI, and AscI sites, then the UBQ3′ and left border sequences. Either Luciferase or C3GFP was then introduced into this vector as NcoI to AscI fragments to create the basic reporter vectors. The alternative test promoters were then used to replace the SVBV promoter as either BamHI to PstI or BamHI to NcoI fragments. This resulted in the creation of a series of constructs for testing SVBV and SVBV derivatives vs. established strong constitutive promoter elements.

C. Assessment of promoter activity

To assess promoter activity we employed two assay systems. One is a transient expression assay in Nicotiana benthamiana leaves and the second is a tobacco hairy root assay. Both assays require the use of Agrobacterium to transfect plant cells.

For the transient expression assay, T-DNA constructs containing the expression cassette of interest were introduced into A. tumefaciens C58. The recombinant agrobacteria were then injected into the interstitial space of the young leaves on the plant and allowed to co-cultivate for two to five days. After this time period the infected material was processed for the reporter assay. GFP expression was observed with a hand-held UV lamp and a Nikon TE300 stereomicroscope. Results are presented in Tables I and 2.

For analysis of stable expression a tobacco hairy root culture system was used. As for the transient system, the test T-DNA vector was first introduced into an appropriate Agrobacteriun strain, in this case A. rhizogeiies K599. Plasmids were introduced into A. rhizogenes K599 by routine electroporation. After electropolation, cells were incubated with shaking for 1 h at 28° C. and plated on LB agar medium. After incubation at 28° C. for 36 h, colonies were picked and plasmid was extracted from randomly selected clones and retransformed into E. coli to assess the fidelity of the transformation process.

Axillary buds of Nicotiana tabacum L. Xanthi were sub-cultured on half-strength Murashige and Skoog (MS) medium with sucrose (1.5%) and agar (1.2%) medium under 16-h light (35-42 μEinsteins m⁻²s⁻¹, cool white fluorescent lamps) at 23° C. every 2-3 weeks. Young leaves were excised from the plant after 2-3 weeks of subculture and were cut into 3×3 mm segments. The pieces were then fully submerged in 20 ml of overnight cultured bacterial suspension (OD 1.0 at 600 nm) in 100×25 mm Petri dishes for 30 min, blotted with autoclaved filter paper, then placed on co-cultivation medium composed of MS solid medium (pH 5.2) with 0.1 mg/l, 1% glucose, and 0.3% Gelrite and incubated as described above. After 3 days of co-cuitivation, 10-20 segments were transferred to basal root induction (BRI) medium. The experiment was repeated at least 2 times with 60 to 80 leaf explants inoculated each time with A. rhizogenes with and without a T-DNA vector.

Once the test strains are created they are co-cultivated with N. tabacum explant tissue and then plated on medium to encourage rooting to occur. After a period of two to three weeks, roots are plentiful and can be assayed for reporter activity.

Transient expression results show that the basic SVBV promoter has comparable to slightly reduced activity relative to the 35S promoter (Tables 1 and 2). However, both of these promoters appear much weaker than their enhanced versions (Table 2). Thus, it appears that duplication of the enhancer region in the SVBV promoter resulted in a dramatic increase in activity. From this experiment it is estimated that the activity of the enhanced SVBV derivatives arc within 75-90% of the d35S and dMMV promoters. More importantly, the leSVBV promoter derivative has equal to or greater activity to that of the d35S promoter in tobacco hairy roots. This is an important observation since this is stably transformed tissue having one to four copies of a transgene as compared to many in a transient expression system. Therefore, this result is particularly relevant in assessing the utility of these promoter derivatives for expression of chimeric genes in transgenic crop plants.

TABLE 1
Measurement of promoter activity by transient
expression of Firefly Luciferase in Nicotiana benthamiana leaves

| Construct | Promoter | Luciferase Specific Activity |
|---|---|---|
| pMAXY2759 | dMMV | 6077 |
| pMAXY2761 | 35S | 3780 |
| pMAXY2762 | SVBV | 2760 |

TABLE 2
Estimation of promoter activity by transient
expression of GFP in Nicotiana benthamiana leaves

| Construct | Promoter | Relative GFP expression |
|---|---|---|
| pMAXY2754 | dMMV | +++++ |
| pMAXY3704 | 35S | + |
| pMAXY3705 | SVBV | + |
| pMAXY3758 | leSVBV | ++++ |
| pMAXY3759 | seSVBV | ++++ |
| pMAXY3760 | d35S | +++++ |

TABLE 3
Relative expression of GFP in tobacco hairy root cultures

| Construct | Promoter | Relative GFP expression |
|---|---|---|
| pMAXY2754 | dMMV | +++++ |
| pMAXY3704 | 35S | +++ |
| pMAXY3705 | SVBV | ++ |
| pMAXY3758 | leSVBV | ++++ |
| pMAXY3759 | seSVBV | +++ |
| pMAXY3760 | d35S | ++++ |

While the foregoing invention has been described in some detail for purposes of clarity and understanding, it will be clear to one skilled in the art from a reading of this disclosure that various changes in form and detail can be made without departing from the true scope of the invention. For example, all the techniques and materials described above can be used in various combinations. All publications and patent documents cited in this application are incorporated by reference in their entirety for all purposes to the same extent as if each individual publication or patent document were so individually denoted.

What is claimed is:

1. An isolated or recombinant nucleic acid molecule comprising a promoter operably linked to a heterologous polynucleotide, wherein the promoter comprises a nucleotide sequence that is at least about 70% identical to a nucleotide sequence selected from SEQ ID NOS: 1-4.

2. The nucleic acid molecule of claim 1, wherein the promoter specifically hybridizes to a nucleic acid comprising a nucleotide sequence selected from SEQ ID NOS: 1-4, or a complement thereof.

3. The nucleic acid molecule of claim 2, wherein the promoter comprises a nucleotide sequence that is at least about 80% identical to a nucleotide sequence selected from SEQ ID NOS: 1-4.

4. The nucleic acid molecule of claim 3, wherein the promoter comprises a nucleotide sequence that is at least about 90% identical to a nucleotide sequence selected from SEQ ID NOS: 1-4.

5. The nucleic acid molecule of claim 4, wherein the promoter comprises a nucleotide sequence selected from SEQ ID NOS: 1-4.

6. An isolated or recombinant nucleic acid molecule comprising a promoter operably linked to a heterologous polynucleotide, wherein the promoter comprises a nucleotide sequence that is at least 90% identical to 100 contiguous nucleotides in a nucleotide sequence selected from SEQ ID NOS: 1-4.

7. An isolated or recombinant nucleic acid molecule comprising a promoter operably linked to a heterologous polynucleotide, wherein the promoter comprises at least 20 contiguous nucleotides in a nucleotide sequence selected from SEQ ID NOS: 1-4.

8. The nucleic acid molecule of claim 7, wherein the promoter comprises at least 40 contiguous nucleotides in a nucleotide sequence selected from SEQ ID NOS: 1-4.

9. The nucleic acid molecule of claim 6, wherein the promoter is derived from SVBV.

10. The nucleic acid molecule of claim 6, wherein the promoter is derived from SVBV Strain E3.

11. The nucleic acid molecule claim 6, wherein the heterologous polynucleotide encodes a polypeptide.

12. The nucleic acid molecule of claim 6, wherein the heterologous polynucleotide encodes an antisense RNA.

13. The nucleic acid molecule of claim 6, further comprising a transcription termination signal.

14. The nucleic acid molecule of claim 6, wherein the nucleic acid molecule is a plasmid suitable for transfection of a plant cell.

15. The nucleic acid molecule of claim 14, wherein the plasmid comprises a selectable marker gene and Agrobacterium border sequences.

16. The nucleic acid molecule of claim 6, wherein the promoter comprises two or more enhancer elements.

17. The nucleic acid molecule of claim 6, wherein the promoter is chimeric.

18. The nucleic acid molecule of claim 17, wherein the chimeric promoter comprises a minimal promoter region derived from SVBV.

19. The nucleic acid molecule of claim 17, wherein the chimeric promoter comprises an enhancer element derived from SVBV.

20. The nucleic acid molecule of claim 19, wherein the chimeric promoter comprises two or more enhancer element derived from SVBV.

21. The nucleic acid molecule of claim 6, wherein the nucleic acid molecule is an expression cassette.

22. A host cell transfected with the nucleic acid molecule of claim 21.

23. The host cell of claim 22, wherein the host cell is a plant cell.

24. The host cell of claim 23, wherein the cell is present within a plant.

25. A transgenic plant comprising the nucleic acid molecule of claim 21.

26. The transgenic plant of claim 25, wherein the plant is a monocot.

27. The transgenic plant of claim 25, wherein the plant is a dicot.

28. A method of expressing a heterologous polynucleotide in a plant cell, the method comprising:
(i) providing an expression cassette comprising a promoter operably linked to the heterologous polynucleotide, wherein the promoter comprises a nucleotide sequence that is at least 90% identical to 100 contiguous nucleotides in a nucleotide sequence selected from SEQ ID NOS: 1-4.; and
(ii) introducing the expression cassette into a plant cell, wherein the heterologous polynucleotide is expressed.

29. The method of claim 28, wherein the plant cell is present within a plant.

30. The method of claim 28, wherein Agrobacterium is used to introduce the nucleic acid molecule into the cell.


SEQUENCE LISTING (10 sequences)

SEQ ID NO: 1 - DNA, 476 bp, Strawberry Vein Banding Virus (SVBV) Strain E3
aactatgctgatgacaagataattctaataagcaattattcagaattaatcaaggagaaagaattaataactctttcagaatatgaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaacc

SEQ ID NO: 2 - DNA, 518 bp, Strawberry Vein Banding Virus (SVBV) Strain E3
ggatcccccagcttaactatgctgatgacaagataattctaataagcaattattcagaattaatcaaggagaaagaattaataactctttcagaatatgaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccggatcgggctgcaggtctctaaccatgg

SEQ ID NO: 3 - DNA, 651 bp, Strawberry Vein Banding Virus (SVBV) Strain E3
ggatccgctttacaagtggccacctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccggatc

SEQ ID NO: 4 - DNA, 701 bp, Strawberry Vein Banding Virus (SVBV) Strain E3
gatccgctttacaagtggccacctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccggatcgggctgcag

SEQ ID NO: 5 - DNA, 35 bp, Artificial Sequence
  Primer
cacacggatccagaagaccaaagggcaattgagac

SEQ ID NO: 6 - DNA, 64 bp, Artificial Sequence
  Primer
acaaactgcagagagagactggtgatttcagcgtgtcctctccaaatgaaatgaacttccttat

SEQ ID NO: 7 - DNA, 35 bp, Artificial Sequence
  Primer
acacaggatccgcgtattggctagagcagcttgcc

SEQ ID NO: 8 - DNA, 40 bp, Artificial Sequence
  Primer
tctctccatggagagatagatttgtagagagagactggtg

SEQ ID NO: 9 - DNA, 22 bp, Artificial Sequence
  Primer
caaagaagaagcttaactatgc

SEQ ID NO: 10 - DNA, 28 bp, Artificial Sequence
  Primer
tgcgggatccggtttgtaagcagggagg